(19) United States
(12) Patent Application Publication
(10) Pub. No.: US 2026/0033785 A1
(43) Pub. Date: Feb. 5, 2026

(54) WEIGHT MEASUREMENT APPARATUS, BIOLOGICAL INFORMATION PROCESSING APPARATUS, AND BIOLOGICAL INFORMATION PROCESSING METHOD

(71) Applicant: CANON MEDICAL SYSTEMS CORPORATION, Otawara-shi (JP)

(72) Inventors: Yohei MURAGUCHI, Nasushiobara (JP); Kosuke ARITA, Kawasaki (JP)

(73) Assignee: CANON MEDICAL SYSTEMS CORPORATION, Otawara-shi (JP)

(21) Appl. No.: 19/279,749

(22) Filed: Jul. 24, 2025

(30) Foreign Application Priority Data
Jul. 30, 2024 (JP) 2024-123081

(51) Int. Cl. A61B 5/00; G01G 19/44

(52) U.S. Cl. CPC A61B 5/7246; A61B 5/7264; A61B 5/7275; A61B 5/742; G01G 19/44

(57) ABSTRACT

A weight measurement apparatus of the embodiment includes processing circuitry. A weight measurement apparatus includes the processing circuitry configured to acquire a body weight of a subject and biological information other than the body weight, calculate a degree of abnormality of the body weight based on a predetermined threshold, calculate a degree of abnormality of a correlation based on a correlation relationship between the body weight and the biological information, and output information indicating the degree of abnormality in the body weight and information indicating a degree of deviation between the correlation relationship used in the calculating of the degree of abnormality in the correlation and a reference correlation relationship.

### CROSS-REFERENCE TO RELATED APPLICATIONS

This application is based upon and claims the benefit of priority from Japanese Patent Application No. 2024-123081, filed on Jul. 30, 2024; the entire contents of which are incorporated herein by reference.

### FIELD

Embodiments described herein relate generally to a weight measurement apparatus, a biological information processing apparatus, and a biological information processing method.

### BACKGROUND

In the related art, it has been known to remotely monitor the state of a subject based on a blood pressure, a heart rate, and other vitals. In such monitoring, the degree of abnormality in subject data is calculated by comparing the distribution of data in the normal case group with the subject data for each of various vitals, and information based on this degree of abnormality is presented to a physician or other medical professionals.

### BRIEF DESCRIPTION OF THE DRAWINGS

FIG. 1 is a block diagram illustrating an example of a configuration of a biological information processing apparatus according to a first embodiment;

FIG. 2 is a diagram for describing an example of a process of calculating the precision matrix according to the first embodiment;

FIG. 3 is a diagram for describing an example of the correlation relationship between vital sign data with different types according to the first embodiment;

FIG. 4 is a diagram for describing an example of the normalization of vital sign data according to the first embodiment;

FIG. 5 is a diagram for describing an example of a process of calculating the degree of abnormality for each piece of vital sign data according to the first embodiment;

FIG. 6 is a diagram for describing an example of a process of calculating the degree of abnormality in the correlation relationship between vital sign data with different types according to the first embodiment;

FIG. 7 is a diagram for describing an example of a process of generating information on findings according to the first embodiment;

FIG. 8 is a diagram for describing an example of a process of generating causal information and intervention candidate information according to the first embodiment;

FIG. 9 is a diagram for describing an example of a process of generating causal information and intervention candidate information according to the first embodiment;

FIG. 10 is a diagram for describing an example of a process of presenting the degree of abnormality in the correlation relationship between vital sign data with different types according to the first embodiment;

FIG. 11 is a flowchart illustrating an example of processing performed by the biological information processing apparatus according to the first embodiment;

FIG. 12 is a block diagram illustrating an example of the configuration of a biological information processing apparatus according to a second embodiment;

FIG. 13 is a flowchart illustrating an example of processing performed by the biological information processing apparatus according to the first embodiment;

FIG. 14 is a diagram for describing an example of a process of presenting the degree of abnormality in the correlation relationship between the vital sign data with different types according to a first modification;

FIG. 15 is a diagram for describing an example of a process of presenting the degree of abnormality in the correlation relationship between the vital sign data with different types according to the first modification; and

FIG. 16 is a diagram for describing an example of a process of presenting the degree of abnormality in the correlation relationship between the vital sign data with different types according to the first modification.

### DETAILED DESCRIPTION

A weight measurement apparatus of the embodiment includes processing circuitry. A weight measurement apparatus includes the processing circuitry configured to acquire a body weight of a subject and biological information other than the body weight, calculate a degree of abnormality of the body weight based on a predetermined threshold, calculate a degree of abnormality of a correlation based on a correlation relationship between the body weight and the biological information, and output information indicating the degree of abnormality in the body weight and information indicating a degree of deviation between the correlation relationship used in the calculating of the degree of abnormality in the correlation and a reference correlation relationship.

The following is a detailed description of embodiments of a weight measurement apparatus, a biological information processing apparatus, and a biological information processing method, with reference to the accompanying drawings.

### First Embodiment

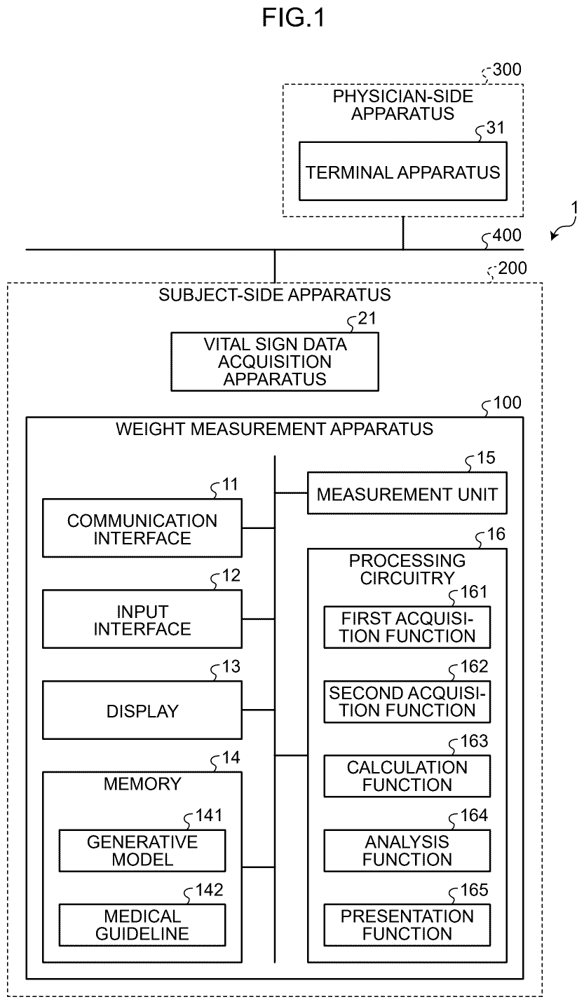

FIG. 1 is a block diagram illustrating an example of the configuration of a weight measurement apparatus 100 according to the first embodiment. Here, FIG. 1 illustrates an example of a diagnostic support system 1 that includes the weight measurement apparatus 100. For example, as illustrated in FIG. 1, the diagnostic support system 1 includes a subject-side apparatus 200 including the weight measurement apparatus 100, and a physician-side apparatus 300, each apparatus being communicatively connected via a network 400.

In the diagnostic support system 1 illustrated in FIG. 1, a single subject-side apparatus 200 and a single physician-side apparatus 300 are illustrated, but a plurality of the subject-side apparatuses 200 and a plurality of the physician-side apparatuses 300 may be connected to the network 400.

In the diagnostic support system 1 illustrated in FIG. 1, only the subject-side apparatus 200 and the physician-side apparatus 300 are illustrated, but various other apparatuses and systems may be connected to the network 400. For example, for the diagnostic support system 1, a data storage apparatus that stores various data on a subject may be connected to the network 400.

The subject-side apparatus 200 includes the weight measurement apparatus 100 and a vital sign data acquisition apparatus 21, and is operated by a subject (patient). The weight measurement apparatus 100 and the vital sign data acquisition apparatus 21 are connected to each other by near field communication or other means to enable mutual communication.

The weight measurement apparatus 100 is an apparatus that measures a weight of the subject and performs various processes using the measured weight of the subject. The weight measurement apparatus 100 is described below.

The vital sign data acquisition apparatus 21 includes various sensors, and acquires and transmits vital sign data of the subject to the weight measurement apparatus 100. For example, the vital sign data acquisition apparatus 21 acquires and transmits vital sign data such as heart rate, pulse rate, blood pressure, electrocardiogram, respiratory status, exercise status (number of steps, time, and the like), body temperature, and blood oxygen saturation to the weight measurement apparatus 100.

The vital sign data acquisition apparatus 21 is implemented as, for example, a wearable apparatus, a biosensor, or a non-contact sensor. Examples of the wearable apparatus include a wristwatch type, an eyeglass type, a ring type, a shoe type, a pocket type, a pendant type, a diaper type, an electroencephalograph type, and the like. Examples of the biosensor also include a simple blood glucose meter and an antigen-antibody testing apparatus (simple urinalysis apparatus), and the like. Examples of the non-contact sensor include a millimeter wave radar or the like.

The vital sign data acquisition apparatus 21 may also change a timing of transmitting vital sign data to the weight measurement apparatus 100 according to the type of vital sign data. For example, the vital sign data acquisition apparatus 21 may transmit measurement results to the weight measurement apparatus 100 in real time for ones that can be measured constantly, such as heart rate.

For example, the vital sign data acquisition apparatus 21 may measure the vitals of the subject at a predetermined time for one such as blood pressure, which is difficult to measure in real time, and transmit the measurement results to the weight measurement apparatus 100 each time the measurement result is output.

The physician-side apparatus 300 includes a terminal apparatus 31 and is operated by a physician who examines the subject. The physician-side apparatus 300 may be an apparatus that can be operated by non-physician healthcare professionals other than physicians. In this case, information that can be viewed, operations that can be performed, and the like may be defined according to the job types of the non-physician healthcare professionals.

The terminal apparatus 31 is an apparatus operated by the physician. The terminal apparatus 31 receives various pieces of information indicating the state of the subject from a biological information processing apparatus 500, and displays the various pieces of information received from the biological information processing apparatus 500 on its own display or outputs the various pieces of information as audio information.

The terminal apparatus 31 also accepts various operations via its own input interface. For example, the terminal apparatus 31 that accepts various operation inputs from the physician and transmits the various operation inputs to the biological information processing apparatus 500 is implemented as, for example, a PC, a tablet PC, PDA, a mobile phone (smartphone, and the like).

Hereinafter, the weight measurement apparatus 100 will be described. For example, the weight measurement apparatus 100 performs various processes to assist the physician in diagnosing the subject. Specifically, the weight measurement apparatus 100 presents information indicating abnormality in the correlation relationship between vital sign data of the subject based on the measured weight of the subject (an example of vital sign data) and a plurality of vital sign data acquired by the vital sign data acquisition apparatus 21.

The weight measurement apparatus 100 includes, as illustrated in FIG. 1, a communication interface 11, an input interface 12, a display 13, a memory 14, a measurement unit 15, and processing circuitry 16.

The communication interface 11 controls transmission and communication of various data transmitted and received between the weight measurement apparatus 100 and each apparatus connected via the network 400.

Specifically, the communication interface 11 is connected to the processing circuitry 16 and transmits data received from each apparatus on the network 400 to the processing circuitry 16 or data received from the processing circuitry 16 to each apparatus on the network 400. For example, the communication interface 11 is implemented as a network card, network adapter, network interface controller (NIC), or the like.

The input interface 12 accepts input operations of various instructions and various pieces of information from an operator. Specifically, the input interface 12 is connected to the processing circuitry 16, converts the input operations received from the operator into electrical signals and transmits the signals to the processing circuitry 16.

For example, the input interface 12 can be implemented as a trackball, a switch button, a touchpad for input operation by touching an operation surface, a touchscreen in which a display screen and a touchpad are integrated, a non-contact input interface using optical sensors, and a voice input interface, or the like.

In the present specification, the input interface 12 is not limited only to those with physical operating components. For example, processing circuitry for electrical signals that receives electrical signals corresponding to input operations from an external input apparatus installed separately from the apparatus and transmits these electrical signals to a control circuit is also included in the example of the input interface 12.

The display 13 displays various pieces of information and data. Specifically, the display 13 is connected to the processing circuitry 16 and displays the measurement results of the weight of the subject received from the processing circuitry 16 and other vital sign data acquired from the vital sign data acquisition apparatus 21. The display 13 is implemented as a liquid crystal display (LCD) or the like.

The memory 14 stores various data and computer programs. For example, the memory 14 stores vital sign data transmitted periodically from the vital sign data acquisition apparatus 21. Only the latest vital sign data transmitted from the vital sign data acquisition apparatus 21 may be stored in the memory 14, or may be retained in the memory 14 until the predetermined period elapses.

For example, the memory 14 stores a generative model 141 and a medical guideline 142. The generative model 141 and the medical guideline 142 will be described below. Specifically, the memory 14 is connected to the processing circuitry 16 and stores data received from the processing circuitry 16, or reads and transmits the stored data to the processing circuitry 16.

For example, the memory 14 is implemented as a semiconductor memory element such as a read only memory (ROM), a random access memory (RAM), a flash memory, or the like, a hard disk drive (HDD), a solid state drive (SSD), an optical disk, and the like. The memory 14 may be implemented as a cloud computer connected to the biological information processing apparatus 500 via the network 400.

The measurement unit 15 measures the weight of the subject as one of the vital sign data. The measurement unit 15 includes a weight sensor. Specifically, the measurement unit 15 is connected to the processing circuitry 16 and transmits the measurement results of the weight of the subject to the processing circuitry 16. The measurement unit 15 may include the weight sensor and electrodes. In this case, the measurement unit 15 may measure weight and body fat percentage as the vital sign data.

The processing circuitry 16 controls the weight measurement apparatus 100 in an overall manner. Specifically, the processing circuitry 16 performs various processes to assist the physician in diagnosing the subject. For example, the processing circuitry 16 controls data exchange with apparatuses on the network 400, data storage in the memory 14, and various processes using the data.

For example, as illustrated in FIG. 1, in the present embodiment, the processing circuitry 16 performs a first acquisition function 161, a second acquisition function 162, a calculation function 163, an analysis function 164, and a presentation function 165.

The first acquisition function 161, together with the second acquisition function 162, acquires a plurality of vital sign data (vital sign measurement values) with different types about the subject. The vital sign data is an example of biological information.

Specifically, the first acquisition function 161 acquires the weight measurement value of the subject transmitted from the measurement unit 15 to the processing circuitry 16. In a case where the measurement unit 15 is capable of measuring body fat percentage, the first acquisition function 161 may acquire the body fat percentage measurement value of the subject together with the weight measurement value of the subject.

The second acquisition function 162 acquires, together with the first acquisition function 161, the vital sign data with different types about the subject.

For example, an acquisition function 551 acquires vital sign data that is periodically acquired by the vital sign data acquisition apparatus 21 from the vital sign data acquisition apparatus 21. Specifically, the second acquisition function 162 acquires vital sign data from the vital sign data acquisition apparatus 21, except for the weight measurement value of the subject.

In a case where the first acquisition function 161 has acquired the body fat percentage measurement value of the subject, the second acquisition function 162 may acquire the vital sign data from the vital sign data acquisition apparatus 21, except for the weight measurement value and the body fat percentage measurement value of the subject.

The calculation function 163 calculates the degree of abnormality in first vital sign data included in the vital sign data with different types from each other based on the correlation relationship with vital sign data other than the first vital sign data. The first vital sign data is an example of first biological information. The vital sign data other than the first vital sign data is also an example of second biological information.

For example, the calculation function 163 calculates the degree of abnormality for each piece of vital sign data (first vital sign data) acquired by the first acquisition function 161 and the second acquisition function 162, as well as the degree of abnormality in the correlation relationship between the vital sign data with different types.

Specifically, the calculation function 163 collects the vital sign data with different types from each other from a plurality of subjects with each piece of vital sign data in the normal range. The calculation function 163 calculates the degree of abnormality for each piece of vital sign data of a target subject to be diagnosed based on the collected vital sign data.

As an example, the calculation function 163 calculates the degree of abnormality for each piece of vital sign data from the following Formula (1).

$${α}_{i} ( x ) = - ln {x}_{i} \vert {x}_{- i} . D = \frac{1}{2} ln \frac{2 π}{{A}_{i , j}} {( {\sum }_{j = 1}^{M} {A}_{i , j} {x}_{j} )}^{2} \qquad ( 1 )$$

Here, λ represents the precision matrix, x represents a value of each piece of normalized vital sign data, and i and j represent the types of vital sign data. The precision matrix represents the direct dependence (correlation relationship) between the vital sign data with different types and can be calculated by the following Formula (2).

$$Λ = {Σ}^{- 1} \qquad ( 2 )$$

Here, Σ represents the variance-covariance matrix, which contains information indicating the variability of each type of vital sign data and the correlation relationship between vital signs.

In the present embodiment, the calculation function 163 calculates λ from the vital sign data with different types from each other, which is collected from the subjects with each piece of vital sign data in the normal range. The calculation function 163 derives the correlation relationship between the vital sign data with different types from the λ calculation result.

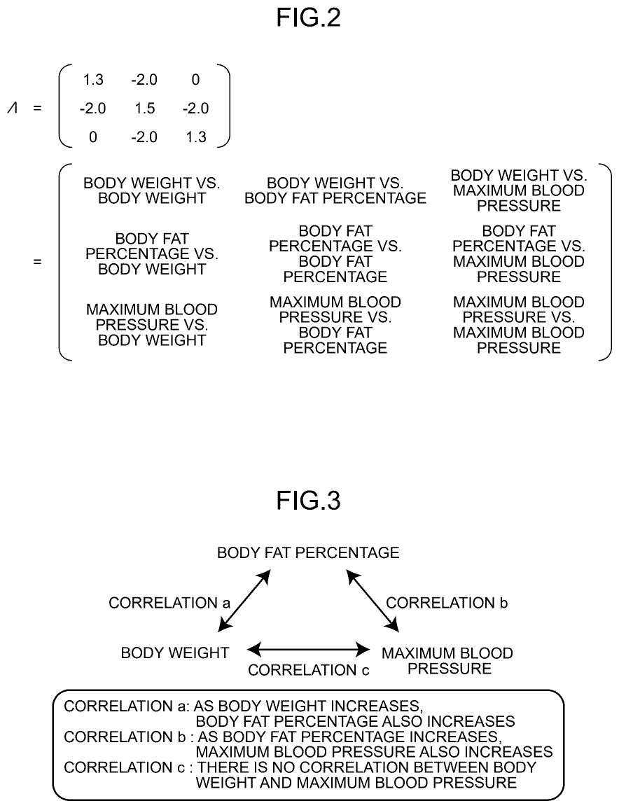

Here, FIG. 2 illustrates an example of a process of calculating the precision matrix. FIG. 2 illustrates an example of λ calculated from the body weight, body fat percentage, and maximum blood pressure data collected from the subjects with each type of vital sign data in the normal range.

A illustrated in FIG. 2 contains information indicating the variability of the data for body weight, body fat percentage, and maximum blood pressure, the correlation relationship between a value of body weight and a value of body fat percentage, the correlation relationship between the value of body weight and a value of maximum blood pressure, and the correlation relationship between the value of body fat percentage and the value of maximum blood pressure. The calculation function 163 derives the correlation relationship between the body weight, body fat percentage, and maximum blood pressure from the λ calculation result.

FIG. 3 is a diagram for describing an example of the correlation relationship between the vital sign data with different types. Since λ_{i,j}<0 represents a positive correlation and λ_{i,j}>0 represents a negative correlation, the calculation function 163 can derive, from the λ calculation result in FIG. 2, “As the body weight increases, the body fat percentage also increases (correlation a)”, “As the body fat percentage increases, the maximum blood pressure also increases (correlation b)”, and “There is no correlation between the body weight and the maximum blood pressure (correlation c)” illustrated in FIG. 3.

In addition, in order to calculate each type of vital sign data of the target subject, the calculation function 163 performs a process of normalizing the vital sign measurement value of each vital sign of the target subject. For example, the calculation function 163 performs an operation to convert the vital sign data with different types collected from the subjects into data with a mean of zero and a variance of one. The above description is an example of a method for normalizing the vital sign data with different types, and normalization methods are not limited to the above description.

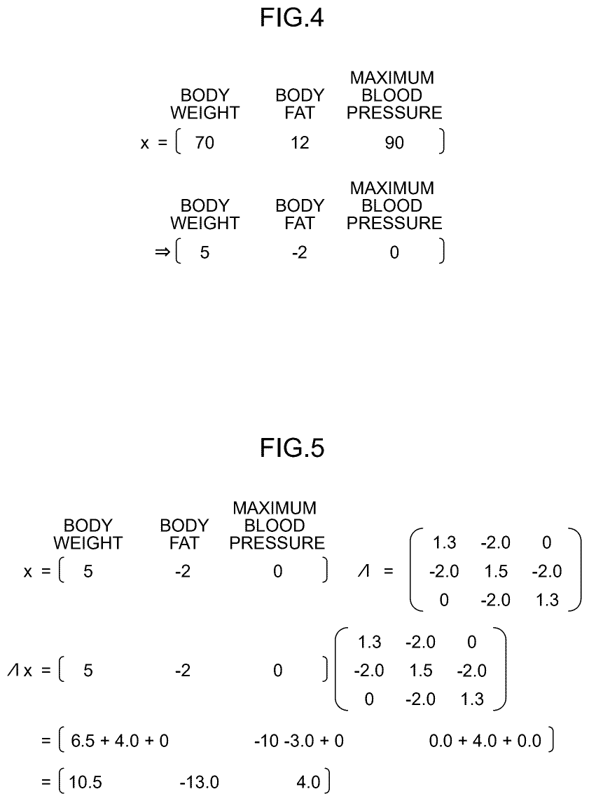

Here, FIG. 4 is a diagram for describing an example of the normalization of vital sign data. In the example in FIG. 4, the body weight, body fat percentage, and maximum blood pressure of the target subject are normalized. The top panel of FIG. 4 represents the weight, body fat percentage, and maximum blood pressure of the target subject before the normalization. In this case, the target subject has a body weight of 70 kg, a body fat percentage of 12%, and a maximum blood pressure of 90 mmHg.

In the example in FIG. 4, the calculation function 163 normalizes the body weight, body fat percentage, and maximum blood pressure of the target subject by performing an operation to convert the vital sign data with different types from each other collected from the subjects into data with a mean of zero and a variance of one.

In the example in FIG. 4, the normalized values for the body weight, body fat percentage, and maximum blood pressure of the target subject are 5 for the body weight, −2 for the body fat percentage, and 0 for the maximum blood pressure. In the example in FIG. 4, the fact that the average is 0 indicates that the subject is heavier, has a lower body fat percentage, and has an average maximum blood pressure.

After normalization of each type of vital sign data of the target subject, the calculation function 163 calculates the degree of abnormality for each piece of vital sign data by substituting the normalized value of each piece of vital sign data of the target subject into the above-described Formula (1).

Here, FIG. 5 is a diagram for describing an example of a process of calculating the degree of abnormality for each piece of vital sign data. As illustrated in Formula (1), the factor that has the strongest influence on the calculation of the degree of abnormality for each piece of vital sign data is the value in the parentheses on the right side of Formula (1). This can be rephrased as follows: as the absolute value in the parentheses on the right side of Formula (1) increases, the degree of abnormality for each piece of vital sign data increases. Therefore, in the example in FIG. 5, for convenience of explanation, only the calculation of the value in parentheses on the right side of Formula (1) will be explained.

In the example in FIG. 5, the value in the parentheses on the right side of Formula (1) can be calculated by determining a product λx of a matrix x indicating the normalized values of the body weight, body fat percentage, and maximum blood pressure of the target subject, and a precision matrix λ. In the example in FIG. 5, the value of λx for the body weight is 10.5, the value of λx for the body fat percentage is −13.0, and the value of λx for the maximum blood pressure is 4.0. In the example in FIG. 5, since the absolute values of λx for the body weight and the body fat percentage are large, it can be seen that the degrees of abnormalities in the body weight and the body fat percentage are high.

In the present embodiment, after the degree of abnormality for each piece of vital sign data of the target subject is calculated, in a case where at least any one of the vital sign data is abnormal, the calculation function 163 calculates the degree of abnormality in the correlation relationship between the vital sign data with different types. For example, in a case where the calculated degree of abnormality for each piece of vital sign data is equal to or more than a threshold, the calculation function 163 calculates the degree of abnormality in the correlation relationship between the vital sign data with different types.

The calculation function 163 may automatically calculate the degree of abnormality in the correlation relationship between the vital sign data with different types after the degree of abnormality for each piece of vital sign data of the target subject is calculated. The calculation function 163 may also calculate the degree of abnormality in the correlation relationship between the vital sign data with different types when an instruction is given by a user.

As an example, the calculation function 163 calculates the degree of abnormality in the correlation relationship between the vital sign data with different types by quantifying the degree of abnormality in the correlation relationship between the vital sign data with different types based on the normalized value of each vital sign measurement value and the calculation result of λ_{i,j} that is also used to calculate the degree of abnormality for each piece of vital sign data.

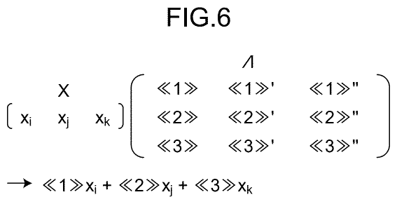

Here, FIG. 6 is a diagram for describing an example of a process of calculating the degree of abnormality in the correlation relationship between the vital sign data with different types. In the example in FIG. 6, the degree of abnormality of a vital sign i of the target subject can be represented by the form of <<1>Xᵢ+<<2>Xⱼ+<<3>Xₖ. As illustrated in FIG. 6, it can be said that the degree of abnormality of the vital i is calculated using the correlation relationship between the vital i and vital j of the target subject, as well as the correlation relationship between the vital i and vital k of the target subject.

In the above Formula, <<2>>xⱼ+<<3>>xₖ represents the term in which <<1>>xᵢ is set to 0 ideally (a case where there is no abnormality at all in the vital i). Therefore, it can be said that <<2>> and <<3>> represent the influence on <<1>>xᵢ.

As the correlation between the vital sign j (or the vital sign k) and the vital sign i is stronger, the influence of <<2>>xⱼ (or <<3>>xₖ) on <<1>>xᵢ is stronger. Thus, the influence on <<1>>xᵢ can be rephrased as the strength of the correlation between xᵢ and xⱼ (or xₖ).

Therefore, for example, the calculation function 163 can quantify the abnormality of the correlation relationship between the vital sign data with different types by determining the difference between <<2>>xⱼ+<<3>xₖ in the ideal case (when <<1>>xᵢ is 0) and the actually calculated <<2>xⱼ+<<3>xₖ based on the strength of the correlation relationship between xᵢ and xⱼ and the strength of the correlation relationship between xᵢ and xₖ.

Specifically, the calculation function 163 can calculate the degree of abnormality in the correlation relationship between the vital sign data with different types using the following Formula (3) or (4).

$$\begin{matrix}{α}_{ij}^{′} = \vert {Λ}_{i , j} \vert * \vert {x}_{i} - {x}_{j} \vert , & {Λ}_{i , j} < 0\end{matrix} \qquad ( 3 )$$

$$\begin{matrix}{α}_{ij}^{′} = \vert {Λ}_{i , j} \vert * \vert {x}_{i} + {x}_{j} \vert , & {Λ}_{i , j} > 0\end{matrix} \qquad ( 4 )$$

The above Formula (3) is used when λ_{i,j}<0, which represents that as xᵢ increases, xⱼ also increases. The above Formula (4) is used when λ_{i,j}>0, which represents that as xᵢ increases, xⱼ decreases.

For example, assuming that λ_{i,j}<0 and that a perfect positive correlation relationship is observed between xᵢ and xⱼ (λ_{i,j}=−1), xᵢ and xⱼ are normalized, and under no abnormality present in the correlation relationship between xᵢ and xⱼ, an increase of 1 in xᵢ is thus expected to result in an increase of 1 in xⱼ.

In such a case, it is assumed that the normalized values of the actual measurement values of xᵢ and xⱼ indicate an increase of 5 in xᵢ is accompanied by an increase of 4 in xⱼ. In this case, the degree of abnormality in the correlation relationship between xᵢ and xⱼ is |−1|×(5−4)=1.

Furthermore, for example, assuming that λ_{i,j}<0 and that xᵢ and xⱼ are almost unrelated (λ_{i,j}=−0.01), an increase in xⱼ can be any value in the case of an increase of 1 in xᵢ.

In such a case, it is assumed that the normalized values of the actual measurement values of xᵢ and xⱼ indicate an increase of 5 in xᵢ is accompanied by an increase of 2 in xⱼ. In this case, the degree of abnormality in the correlation relationship between xᵢ and xⱼ is |−0.01|×(5−2)=0.03.

Thus, in a case where a correlation coefficient between xᵢ and xⱼ is small, the degree of abnormality in the correlation between xᵢ and xⱼ does not increase even though the difference between xᵢ and xⱼ increases. In contrast, in a case where the correlation coefficient between xᵢ and xⱼ is large, the degree of abnormality in the correlation between xᵢ and xⱼ increases even though the difference between xᵢ and xⱼ decreases.

The above-described calculation methods for the degree of abnormality for each piece of vital sign data and the degree of abnormality in the correlation relationship between the vital sign data with different types are examples, and the calculation methods for the degree of abnormality for each piece of vital sign data and the degree of abnormality in the correlation relationship between the vital sign data with different types are not limited thereto. Any method may be used as long as it is capable of calculating an index representing the degree of abnormality for each piece of vital sign data and an index representing the degree of abnormality in the correlation relationship between the vital sign data with different types.

As an example, the calculation function 163 may set a predetermined threshold for each piece of vital sign data and calculate the degree of abnormality for each piece of vital sign data by comparing each vital sign measurement value with the predetermined threshold of each piece of vital sign data.

Return to FIG. 1 to continue the explanation. The analysis function 164 analyzes the calculation result of the degree of abnormality for each piece of vital sign data and the calculation result of the correlation relationship between the vital sign data with different types.

For example, the analysis function 164 generates information on findings indicating findings derived from the degree of abnormality for each piece of vital sign data and the degree of abnormality in the correlation relationship between the vital sign data with different types. As an example, the analysis function 164 generates information on findings based on: the process of calculating the degree of abnormality for each piece of vital sign data and the degree of the correlation relationship between the vital sign data with different types, and the calculation result; and a formulated phrase representing the findings.

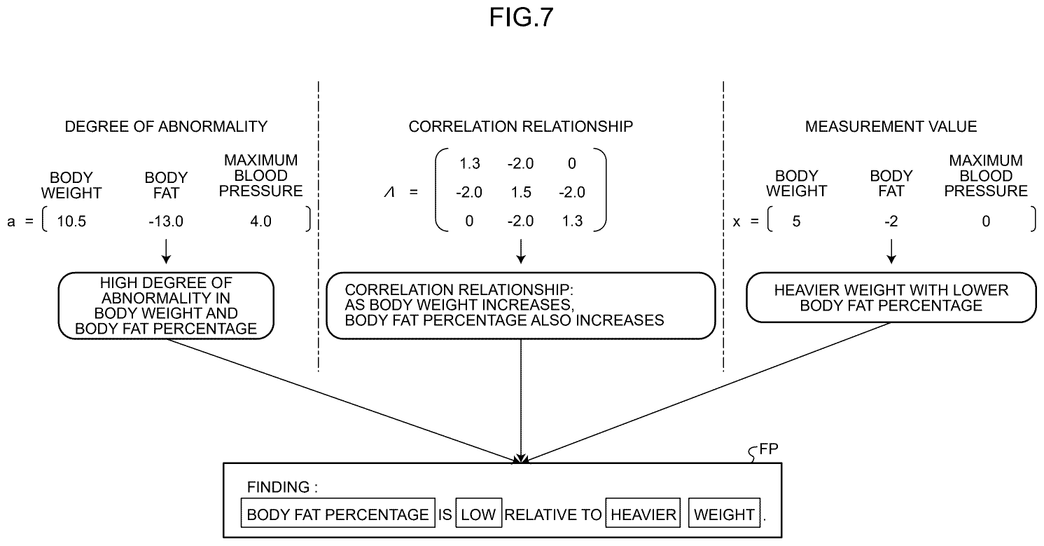

Here, FIG. 7 is a diagram for describing an example of a process of generating information on findings. In the example in FIG. 7, a formulated phrase FP is “(A) is (B) while (C) is (D)”. For example, the formulated phrase FP is stored in the memory 14, or the like. In the example in FIG. 7, the analysis function 164 generates information on findings by populating the placeholders (A) to (D) described above based on the calculation results of the degree of abnormality for each piece of vital sign data and the degree of the correlation relationship between the vital sign data with different types.

In the example in FIG. 7, the analysis function 164 derives a “high degree of abnormality in body weight and body fat percentage” from the degree of abnormality in each of the calculated body weight, body fat percentage, and maximum blood pressure of the target subject.

The analysis function 164 also derives the correlation relationship between the body weight and the body fat percentage, specifically that “as the body weight increases, the body fat percentage also increases” from the calculation result of A, which appears in the process of calculating the degree of abnormality for each piece of vital sign data.

Furthermore, the analysis function 164 derives “heavier weight with lower body fat percentage” from the normalized measurement values of the body weight, body fat percentage, and maximum blood pressure of the target subject, which appear in the process of calculating the degree of abnormality for each piece of vital sign data.

The analysis function 164 populates the placeholders (A) to (D) in the formulated phrase FP based on the above-described information obtained by analyzing the calculation process and calculation result of the degree of abnormality in the correlation relationship between the vital sign data with different types, and generates information on a finding that “(weight) is (heavy) while (body fat percentage) is (low)”.

In addition, a sentence such as “(a symptom) is observed”, may be added to the formulated phrase. In this case, the analysis function 164 may refer to an electronic medical record and the like of the target subject to populate the parentheses with a phrase such as “(exacerbation of heart failure) is observed”. As a result, the analysis function 164 can generate information on findings including information representing symptoms of the target subject.

For example, the analysis function 164 generates, based on the generated information on findings, causal information indicating a cause of abnormality of the correlation relationship between the vital sign data with different types and intervention candidate information indicating candidates for intervention to reduce the abnormality of the correlation relationship. As an example, the analysis function 164 generates the causal information and the intervention candidate information using the generative model 141 and the medical guideline 142 stored in the memory 14.

Here, the generative model 141 is a model for generating causal information and intervention candidate information. For example, the generative model 141 is a large language model (hereafter, also referred to as LLM).

LLM is an AI model pre-trained on large corpora in the field of natural language processing. For example, LLM is a model that is functionalized to generate and output a response sentence in accordance with the meaning of an input sentence when a question or instruction is input as a sentence (prompt).

The medical guideline 142 represents a guideline that summarizes information about the rationale and procedures for medical treatment, including the prevention, diagnosis, treatment, and prognosis prediction of disease. The medical guideline 142 may be stored in the memory 14 separately for each type of disease. The generative model 141 and the medical guideline 142 may be stored on an external server or the like different from the weight measurement apparatus 100.

Here, the information on findings, the causal information, and the intervention candidate information are included in information that assists the physician in diagnosing the target subject. For this reason, in the following description, the information on findings, the causal information, and the intervention candidate information are also referred to as diagnostic support information.

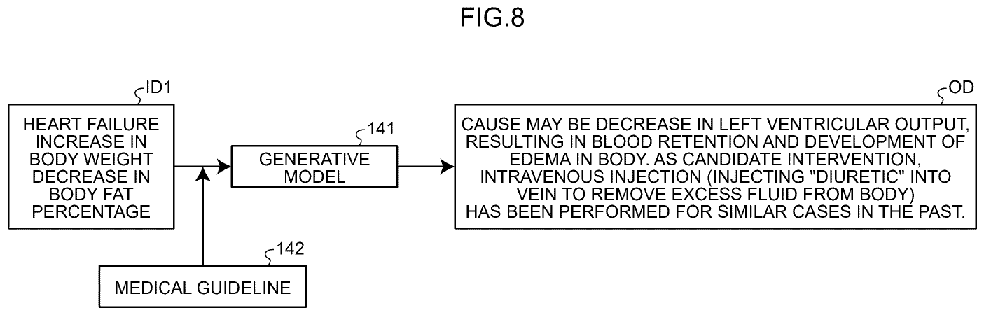

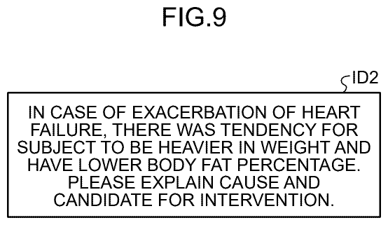

Hereinbelow, an example of a process of generating causal information and intervention candidate information by the analysis function 164 will be described with reference to FIGS. 8 and 9. FIGS. 8 and 9 are diagrams for describing an example of a process of generating causal information and intervention candidate information.

As an example, the analysis function 164 extracts an input word ID1 serving as the basis for a sentence to be input to the generative model 141, from the generated information on findings of the target subject or electronic medical records of the target subject. The analysis function 164 may convert the expression of a word extracted as the input word ID1 into the general expression or perform other processing by using known natural language processing techniques, or the like.

In the example in FIG. 8, the analysis function 164 extracts, from the generated information on findings and the like, “heart failure” indicating a disease from which the target subject is suffering, “increase in body weight” and “decrease in body fat percentage” indicating the abnormality of vital sign data of the target subject.

In the example in FIG. 8, the analysis function 164 refers to the medical guideline 142 and performs keyword search, vector search, or the like for the input word ID1 (“heart failure”, “increase in body weight”, and “decrease in body fat percentage”). Based on the search results, the analysis function 164 generates input sentences to be input into the generative model 141 to generate causal information and intervention candidate information.

As described above, since searching the medical guideline 142 is performed and the sentences to be input to the generative model 141 are generated based on the search results, it is possible to prevent the generative model 141 from generating causal information and intervention candidate information whose contents deviate from the medical guideline 142.

The analysis function 164 inputs the generated input sentences to the generative model 141. The analysis function 164 generates response sentences output from the generative model 141 as causal information and intervention candidate information.

In the example in FIG. 8, the generative model 141 generates and outputs an output sentence OD “The cause may be a decrease in left ventricular output, resulting in blood retention and the development of edema in the body. As a candidate intervention, intravenous injection (injecting a “diuretic” into a vein to remove excess fluid from the body) has been performed for similar cases in the past”.

In the above-described case, the analysis function 164 generates, based on the output sentence OD, causal information such as “The cause may be a decrease in left ventricular output, resulting in blood retention and the development of edema in the body”. Similarly, the analysis function 164 generates intervention candidate information such as “As a candidate intervention, intravenous injection (injecting a “diuretic” into a vein to remove excess fluid from the body) has been performed for similar cases in the past”.

In the example in FIG. 8, the analysis function 164 extracts the input word ID1, but as illustrated in FIG. 9, the analysis function 164 may also generate an input sentence ID2 based on the generated information on findings of the target subject and electronic medical records of the target subject.

In the above-described case, the analysis function 164 may refer to the medical guideline 142 and perform keyword search, vector search, or the like for the input sentence ID2, or may not refer to the medical guideline 142 and directly input the generated input sentence ID2 to the generative model 141.

The analysis function 164 may generate causal information and intervention candidate information using only the medical guideline 142.

For example, the analysis function 164 may perform keyword search or vector search for the input word ID1, and present the results to a user such as a physician, by highlighting a part of text data stored as the medical guideline 142 that is presumed to indicate the cause of the abnormality based on the search results. Similarly, the analysis function 164 may present the results to the user by highlighting a part that can be presumed to indicate intervention candidates for reducing the abnormality in a different manner than described above.

The presentation function 165 presents the degree of abnormality in the correlation relationship between the vital sign data with different types for each type of vital sign data other than the first vital sign data.

Here, the degree of abnormality in the correlation relationship between the vital sign data with different types can be referred to as a degree of deviation between a reference correlation relationship and the correlation relationship between vital sign data with different types, derived based on each type of vital sign data of the target subject, by using the correlation relationship between the vital sign data with different types derived based on the vital sign data with different types from each other, collected from the subjects with each piece of vital sign data in the normal range, as the reference correlation relationship.

The above-described calculation function 163 uses λ to calculate the degree of abnormality for each piece of vital sign data, and λ includes information about the correlation relationship between the vital sign data with different types. Furthermore, in the process of calculating the degree of abnormality for each piece of vital sign data, the calculation function 163 performs a calculation that multiplies λ by the normalized value of the measurement value of each vital sign of the target subject.

Therefore, it can be said that the calculation function 163 uses the correlation relationship between the vital sign data with different types of the target subject to calculate the degree of abnormality for each piece of vital sign data.

Therefore, it can be said that the degree of abnormality in the correlation relationship between the vital sign data with different types is an example of information indicating the degree of deviation between the correlation relationship used to calculate the degree of abnormality of the first vital sign data from the reference correlation relationship.

For example, in a case where at least one of the degrees of abnormality of vital sign data is equal to or more than a threshold, the presentation function 165 presents, to the user, the degree of abnormality for each piece of vital sign data and the degree of abnormality in the correlation relationship between the vital sign data with different types as a vertical bar graph by displaying the results on a display (display unit) of the terminal apparatus 31 of the physician-side apparatus 300 or the like.

For example, in a case where at least one degree of abnormality of each type of vital sign data is equal to or more than the threshold, the presentation function 165 informs the user of a warning (alert) according to a disease from which the target subject is suffering or the type of vital sign data that is equal to or more than the threshold.

As an example, in a case where the degree of abnormality of the heart rate of a target subject with heart failure is equal to or more than the threshold, the presentation function 165 warns the user that the heart failure of the target subject may have worsened by displaying a warning message on the display of the terminal apparatus 31 of the physician-side apparatus 300 or the like.

The presentation function 165 may report a warning to the user and may also report the same warning to the target subject via the display 13 of the subject-side apparatus 200 for the target subject.

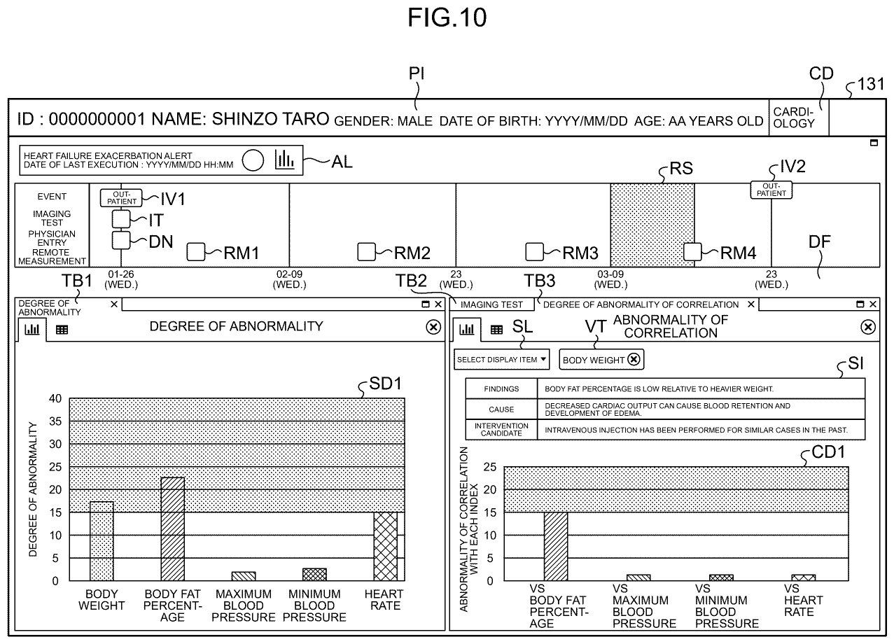

Here, FIG. 10 is a diagram for describing an example of a process of presenting the degree of abnormality in the correlation relationship between vital sign data with different types.

In the example in FIG. 10, a presentation screen 131 for the degree of abnormality in vital sign data includes, as its screen components: a patient information display section PI, a department display section CD, an alert display section AL, a date display section DF, a selection range RS, outpatient icons IV (IV1 and IV2), an imaging test icon IT, a physician entry icon DN, remote measurement icons RM (RM1 to RM4), an abnormality degree tab TB1, an imaging test tab TB2, a correlation abnormality tab TB3, an abnormality degree graph SD1, a selection box SL, a vital sign tab VT, a correlation abnormality degree graph CD1, and a diagnostic support information display section SI.

The patient information display section PI is a section in which patient information about the target subject is displayed. In the example in FIG. 10, the name (Shinzo Taro), gender (male), date of birth (YYYY/MM/DD), and age (AA years old) of the target subject are displayed.

The department display section CD is a display section in which the medical department for the target subject is displayed. In the example in FIG. 10, “Cardiology” is displayed. In a case where the target subject has visited multiple departments, the user may be able to select the department by clicking on the department display section CD.

The alert display section AL is a display section in which alerts are displayed. For example, the content of an alert and the date and time of the most recent alert (date of last execution) are displayed in the alert display section AL. In the example in FIG. 10, the alert display section AL displays that a “Heart failure exacerbation alert” was issued, most recently on YYYY/MM/DD HH:MM.

The date display section DF is a display section in which the date when the medical event regarding the target subject occurred or when the medical event is scheduled to take place is displayed. The selection range RS represents a range of dates selected for viewing medical events. For example, the lower portion of the presentation screen 131 displays information about medical events within the selection range RS.

The outpatient icons IV (IV1, IV2) are icons indicating that an outpatient examination of the target subject has been performed or is scheduled to be performed. In the example in FIG. 10, the outpatient icon IVI indicates that the target subject was treated as an outpatient on January 26 (Wednesday). In addition, the outpatient icon IV2 indicates that the target subject was treated as an outpatient on March 23 (Wednesday).

The imaging test icon IT is an icon indicating that an imaging test has been performed or is scheduled to be performed on the target subject. In the example in FIG. 10, the image test icon IT indicates that an image test was performed on the target subject on January 26 (Wednesday).

The physician entry icon DN is an icon indicating that a physician has made some entry in the electronic medical record of the target subject. In the example in FIG. 10, the imaging test icon IT represents that the physician made some entry in the electronic medical record of the target subject on Wednesday, January 26.

The remote measurement icons RM are icons indicating that vital sign data has been transmitted or will be transmitted from the subject-side apparatus 200.

In the example in FIG. 10, the remote measurement icon RM1 indicates that vital sign data was transmitted from the subject-side apparatus 200 between January 26 (Wednesday) and February 9 (Wednesday). In addition, the remote measurement icon RM2 indicates that vital sign data was transmitted from the subject-side apparatus 200 between February 9 (Wednesday) and February 23 (Wednesday).

Furthermore, the remote measurement icon RM3 indicates that vital sign data was transmitted from the subject-side apparatus 200 of the target subject between February 23 (Wednesday) and March 9 (Wednesday). Furthermore, the remote measurement icon RM3 indicates that vital sign data was transmitted from the subject-side apparatus 200 of the target subject between February 9 (Wednesday) and March 23 (Wednesday).

The abnormality degree tab TB1 is a tab for displaying information on the degree of abnormality for each piece of vital sign data in the selection range RS. For example, when the user clicks on the abnormality tab TB1, an abnormality degree graph SD1 is displayed. The abnormality degree graph SD1 will be described later.

The imaging test tab TB2 is a tab for displaying the results of an imaging test within the selection range RS.

The correlation abnormality tab TB3 is a tab for displaying information on the degree of abnormality between the vital sign data with different types. For example, when the user clicks on the correlation abnormality tab TB2, the selection box SL, the vital sign tab VT, the correlation abnormality degree graph CD1, and the diagnosis support information display section SI are displayed. The selection box SL, vital sign tab VT, correlation abnormality degree graph CD1, and diagnostic support information display section SI are described below.

The abnormality degree graph SD1 is a vertical bar graph representing the degree of abnormality for each piece of vital sign data calculated by the calculation function 163. In the example in FIG. 10, the vertical axis represents the degree of abnormality for each piece of vital sign data, and the horizontal axis represents the types of vital sign data. In the example illustrated in FIG. 10, the vertical bar graph represents the degrees of abnormalities in the body weight, the body fat percentage, the maximum blood pressure, the minimum blood pressure, and the heart rate, indicating that the degrees of abnormalities in the body weight, the body fat percentage, and the heart rate are equal to or more than a threshold.

The selection box SL is a selection box for selecting the type of vital sign data. For example, when the user clicks on the selection box SL, a list for selecting one of the following is displayed: the body weight, the body fat percentage, the maximum blood pressure, the minimum blood pressure, and the heart rate. For example, in a case where the user selects a type of vital sign data, a vital sign tab VT corresponding to the selected vital sign type is displayed.

The vital sign tab VT is a tab for switching the types of vital sign data to be displayed for the degree of abnormality of the correlation relationship.

In the example in FIG. 10, the vital sign tab VT is displayed for body weight. For example, if the user selects body fat percentage by clicking on the selection box SL, an additional Vitals tab VT corresponding to body fat percentage will be displayed, allowing the user to switch the type of vital sign data to be displayed between weight and body fat percentage.

The correlation abnormality degree graph CD1 is a vertical bar graph representing the degree of abnormality in the correlation relationship between the vital sign data with different types calculated by the calculation function 163. In the example in FIG. 10, the vertical axis represents the degree of abnormality in the correlation relationship between the type of vital sign data corresponding to the vital sign tab VT and other types of vital sign data, and the horizontal axis represents other types of vital sign data.

In the example in FIG. 10, the degree of abnormality in the correlation relationship between the body weight and the body fat percentage, the degree of abnormality in the correlation relationship between the body weight and the maximum blood pressure, the degree of abnormality in the correlation relationship between the body weight and the minimum blood pressure, and the degree of abnormality in the correlation relationship between the body weight and the heart rate are represented by the vertical bar graph, and the vertical bar graph indicates that the degree of abnormality in the correlation relationship between the body weight and the body fat percentage is equal to or more than the threshold.

The diagnostic support information display section SI is a display section in which diagnostic support information generated by the analysis function 164 is displayed.

In the example in FIG. 10, in the diagnostic support information display section SI, “body fat percentage is low relative to heavier weight” is displayed as the information on findings. In addition, in the diagnostic support information display section SI, “Decreased cardiac output can cause blood retention and the development of edema.” is displayed as the causal information. In addition, in the diagnostic support information display section SI, “Intravenous injection has been performed for similar cases in the past” is displayed as the intervention candidate information.

Thus, the weight measurement apparatus 100 according to the present embodiment displays not only the degree of abnormality for each piece of vital sign data alone, but also the degree of abnormality of the correlation relationship between the vital sign data with different types. As a result, this facilitates the user's understanding of the condition of the target subject. The weight measurement apparatus 100 according to the present embodiment also displays diagnostic support information. As a result, the user can more readily conduct a diagnosis and make decisions regarding treatment methods for the target subject.

The processing circuitry 16 described above is implemented as, for example, a processor. In that case, each processing function described above is stored in the memory 14 in the form of a computer program executable by a computer. Then, the processing circuitry 16 reads and executes each computer program stored in the memory 14 to implement the function corresponding to each computer program. In other words, the processing circuitry 16 has each processing function illustrated in FIG. 1 with each computer program read out.

The processing circuitry 16 may include a plurality of independent processors, each of which executes a computer program to implement each processing function. The processing functions that the processing circuitry 16 has may be distributed or integrated into a single or a plurality of processing circuitries as appropriate. Each processing function that the processing circuitry 16 has may be implemented in combination of hardware such as circuits and software.

Although the example in which the computer program corresponding to each processing function is stored in a single memory 14 is described herein, the embodiment is not limited thereto. For example, computer programs corresponding to processing functions may be distributed and stored in a plurality of memories, and the processing circuitry 16 may read and execute each program from each memory.

Some of the processing functions that the processing circuitry 16 has may also be implemented by a cloud computer connected to the weight measurement apparatus 100 via the network 400.

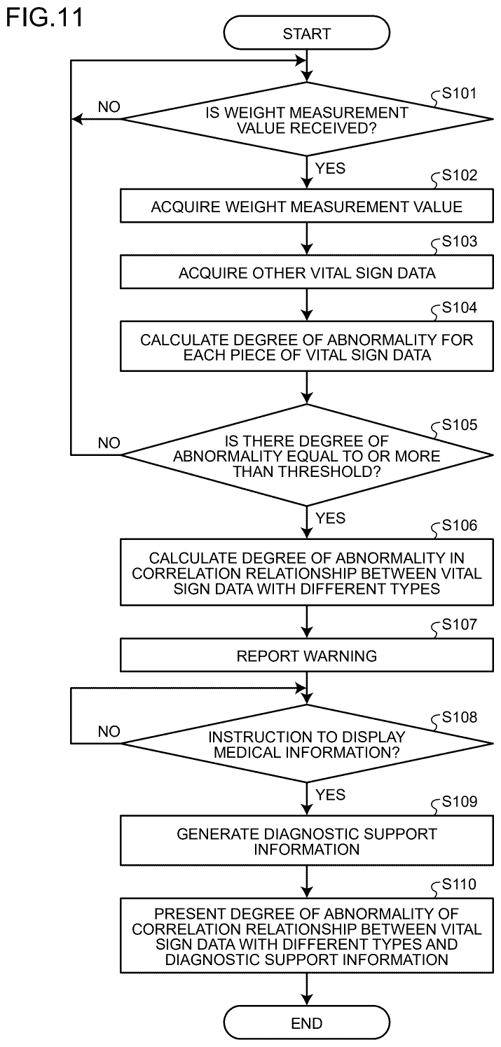

Next, a process executed by the weight measurement apparatus 100 will be described. FIG. 11 is a flowchart illustrating an example of a process executed by the weight measurement apparatus 100.

First, the first acquisition function 161 determines whether a weight measurement value has been received from the measurement unit 15 (step S101). For example, the first acquisition function 161 determines whether the processing circuitry 16 has received a weight measurement value from the measurement unit 15. In a case where no weight measurement value is received (No at step S101), the process of step S101 is repeated.

On the other hand, in a case where the weight measurement value is received (Yes at step S101), the first acquisition function 161 acquires the weight measurement value of the target subject (step S102). For example, the first acquisition function 161 acquires the weight measurement value received from the measurement unit 15 at step S101.

Next, the second acquisition function 162 acquires other vital sign data of the target subject (step S103).

For example, the second acquisition function 162 acquires a plurality of vital sign data with different types, which are periodically transmitted from the vital sign data acquisition apparatus 21 of the subject-side apparatus 200 of the target subject and stored in the memory 14. Specifically, the second acquisition function 162 acquires, from the memory 14, the latest of each type of vital sign data at the time when the measured weight value of the target subject has been acquired at step S101.

Next, the calculation function 163 calculates the degree of abnormality for each piece of vital sign data of the target subject (step S104). For example, the calculation function 163 calculates the degree of abnormality for each piece of vital sign data with respect to the vital sign data with different types acquired at steps S102 and S103. The specific calculation method is as described above, and the description thereof will not be described here.

Next, the calculation function 163 determines whether the degree of abnormality for each piece of vital sign data of the target subject is equal to or more than a threshold (step S105). For example, the calculation function 163 determines whether the degree of abnormality for each piece of vital sign data of the target subject calculated at step S104 is equal to or more than a threshold. In a case where none of them are equal to or more than a threshold (No at step S105), the process returns to S101.

On the other hand, in a case where there is any that is equal to or more than a threshold (Yes at step S105), the calculation function 163 calculates the degree of abnormality in the correlation relationship between the vital sign data with different types of the target subject (step S106). The specific calculation method is as described above, and the description thereof will not be described here.

In FIG. 11, the process at step S106 is described as the process performed after step S105, but the process at step S106 may be performed after step S104 and before the process at step S105. In this case, the process at step S106 will be performed automatically after step S104, regardless of the result of the calculation of the degree of abnormality for each piece of vital sign data of the target subject.

Next, the presentation function 165 reports a warning to the user (step S107). For example, the presentation function 165 executes a process of issuing an alert to the user, the alert including contents dependent on the type of vital data whose degree of abnormality is equal to or more than a threshold, a disease from which the target subject is suffering, and the like. The process at step S107 may be performed after the process at step S105 described above and prior to the process at step S106, or in parallel with the process at step S106.

Next, the analysis function 164 determines whether the user has given an instruction to display medical information regarding medical treatment of the target subject (step S108). Here, in general, in a case where the target subject for whom a warning has been reported visits a medical institution, the user gives an instruction for displaying medical information in order to examine the target subject. In a case where there is no instruction to display medical information (No at step S108), the process at step S108 is repeated.

On the other hand, in a case where there is an instruction to display medical information (Yes at step S108), the analysis function 164 generates diagnostic support information (step S109). The specific generation method is as described above, and the description thereof will not be described here.

In FIG. 11, the process at step S109 is described as the process performed after step S108, but the process at step S109 may be performed after determining Yes at step S105 and before the process at step S108. In this case, the process at step S109 may be performed prior to the process at step S107 or in parallel with the process at step S107.

Next, the presentation function 165 presents the degree of abnormality of correlation relationship between the vital sign data with different types and diagnostic support information to the user (step S110), and ends the present process. For example, the presentation function 165 displays the presentation screen 131 illustrated in FIG. 10 on the display of the terminal apparatus 31 of the physician-side apparatuses 300.

As described above, the weight measurement apparatus 100 according to the present embodiment acquires a plurality of vital sign data with different types for the subject, calculates the degree of abnormality of the first vital sign data of the vital sign data based on the correlation relationship with the types of vital sign data other than the first vital sign data, and presents the degree of abnormality in the correlation relationship between the vital sign data with different types, indicating the degree of deviation between the correlation relationship used to calculate the degree of abnormality and the reference correlation for each type of vital sign data other than the first vital sign data.

As a result, the weight measurement apparatus 100 according to the present embodiment can calculate an index that can identify not only abnormalities in specific vital signs alone, but also abnormalities that take into account the correlation relationship between specific signs and other signs. The weight measurement apparatus 100 according to the present embodiment can also present, to the user, the degree of deviation between the correlation relationship between specific vital signs and other vital signs derived from the vital sign data of the target subject to be diagnosed and the reference correlation (for example, the correlation relationship between specific vital signs and other vital signs derived from the vital sign data of a plurality of target subjects whose vital signs are in the normal range). The presentation of such information makes it easier for the user to understand the state of the target subject, for example, compared to the case where the user is informed of whether specific vital signs alone are abnormal. In other words, the weight measurement apparatus 100 of the present embodiment can assist the user to easily understand the state of the target subject.

The weight measurement apparatus 100 according to the present embodiment also generates information on findings indicating the findings based on the calculated degree of abnormality of each type of vital sign data, and displays the generated information on findings along with the degree of abnormality of each type of vital sign data on a display unit of the terminal apparatus 31 of the physician-side apparatus 300 or the like.

As a result, for example, even a user who is not an expert in the disease from which the target subject is suffering or who has little experience can easily grasp whether the abnormality has occurred in the target subject.

The weight measurement apparatus 100 according to the present embodiment also generates causal information indicating a cause of abnormality in each type of vital sign data, and displays the generated causal information along with the degree of abnormality of each type of vital sign data on the display unit of the terminal apparatus 31 of the physician-side apparatus 300 or the like.

As a result, for example, even a user who is not an expert in the disease from which the target subject is suffering or who has little experience can easily understand the cause of the abnormality that has occurred in the target subject.

The weight measurement apparatus 100 also generates intervention candidate information indicating candidates for intervention to reduce the abnormality occurring in the target subject, and displays the generated intervention candidate information along with the degree of abnormality for each piece of vital sign data on the display unit of the terminal apparatus 31 of the physician-side apparatus 300, or the like.

As a result, even a user who is not an expert in the disease from which the target subject is suffering or who has little experience can easily determine how to deal with the target subject in which the abnormality is occurring.

### Second Embodiment

The first embodiment described above describes the form in which the first acquisition function 161, the second acquisition function 162, the calculation function 163, the analysis function 164, and the presentation function 165 are implemented by the processing circuitry of the weight measurement apparatus 100. A second embodiment describes a form in which functions equivalent to these are implemented by processing circuitry of the biological information processing apparatus that is provided separately from the weight measurement apparatus 100.

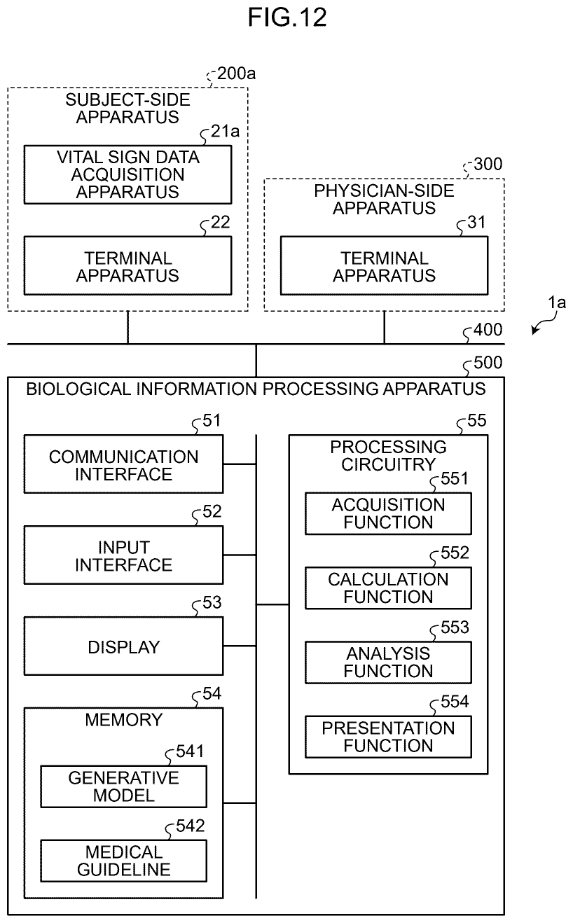

FIG. 12 is a block diagram illustrating an example of the configuration of a biological information processing apparatus 500 according to the second embodiment. Here, FIG. 12 illustrates an example of a diagnostic support system 1a that includes the biological information processing apparatus 500. For example, as illustrated in FIG. 12, the diagnostic support system 1a includes a subject-side apparatus 200a, a physician-side apparatus 300, and the biological information processing apparatus 500, each apparatus being communicatively connected via a network 400.

In the diagnostic support system 1a illustrated in FIG. 12, a single subject-side apparatus 200a and a single physician-side apparatus 300 are illustrated, but a plurality of the subject-side apparatuses 200a and a plurality of the physician-side apparatuses 300 may be connected to the network 400.

In the diagnostic support system 1a illustrated in FIG. 12, only the biological information processing apparatus 500, the subject-side apparatus 200a and the physician-side apparatus 300 are illustrated, but various other apparatuses and systems may be connected to the network 400. For example, for the diagnostic support system 1a, a data storage apparatus that stores various data on a subject may be connected to the network 400.

The subject-side apparatus 200a includes a vital sign data acquisition apparatus 21a and a terminal apparatus 22, and is operated by a subject (patient). The vital sign data acquisition apparatus 21a and the terminal apparatus 22 are connected to each other by near field communication or other means to enable mutual communication.

The vital sign data acquisition apparatus 21a includes various sensors, and acquires and transmits vital sign data of the subject to the terminal apparatus 22. For example, the vital sign data acquisition apparatus 21a acquires and transmits vital sign data such as body weight, body composition, heart rate, pulse rate, blood pressure, electrocardiogram, respiratory status, exercise status (number of steps, time, and the like), body temperature, and blood oxygen saturation to the terminal apparatus 22.

The vital sign data acquisition apparatus 21a is implemented as, for example, a weight sensor (body weight measurement apparatus), an electrode (body composition measurement apparatus), a wearable device, a biosensor, a non-contact sensor, and the like. Examples of the wearable apparatus include a wristwatch type, an eyeglass type, a ring type, a shoe type, a pocket type, a pendant type, a diaper type, an electroencephalograph type, and the like. Examples of the biosensor also include a simple blood glucose meter and an antigen-antibody testing apparatus (simple urinalysis apparatus), and the like. Examples of the non-contact sensor include a millimeter wave radar or the like.

The vital sign data acquisition apparatus 21a may also change a timing of transmitting vital sign data to the terminal apparatus 22 according to the type of vital sign data. For example, the vital sign data acquisition apparatus 21a may transmit measurement results to the terminal apparatus 22 in real time for ones that can be measured constantly, such as heart rate.

For example, the vital sign data acquisition apparatus 21a may measure the vitals of the subject at a predetermined time for one such as blood pressure, which is difficult to measure in real time, and transmit the measurement results to the terminal apparatus 22 each time the measurement result is output.

The terminal apparatus 22 is an apparatus operated by the subject. The terminal apparatus 22 transmits the vital sign data of the subject received from the vital sign data acquisition apparatus 21a to the biological information processing apparatus 500. The terminal apparatus 22 may display various pieces of information received from the biological information processing apparatus 500 on its own display or output the various pieces of information as audio information.

The terminal apparatus 22 also accepts various operations via its own input interface. For example, the terminal apparatus 22 can also accept input operations from a subject about their behavior and psychological state, and transmit the input operations to the biological information processing apparatus 500. The terminal apparatus 22 is implemented by, for example, a PC, a tablet PC, a PDA, a mobile phone (smartphone, and the like), or the like.

In the present embodiment, a case in which the terminal apparatus 22 transmits the vital sign data acquired by the vital sign data acquisition apparatus 21a to the biological information processing apparatus 500 will be described, but the embodiment is not limited thereto. For example, the vital sign data acquisition apparatus 21a may have a communication function with the biological information processing apparatus 500, and the vital sign data acquisition apparatus 21a may communicate with the biological information processing apparatus 500.

The physician-side apparatus 300 includes a terminal apparatus 31 and is operated by a physician who examines the subject. The physician-side apparatus 300 may be an apparatus that can be operated by non-physician healthcare professionals other than physicians. In this case, information that can be viewed, operations that can be performed, and the like may be defined according to the job types of the non-physician healthcare professionals.

The terminal apparatus 31 is an apparatus operated by the physician. The terminal apparatus 31 receives various pieces of information indicating the state of the subject from a biological information processing apparatus 500, and displays the various pieces of information received from the biological information processing apparatus 500 on its own display or outputs the various pieces of information as audio information.

The terminal apparatus 31 also accepts various operations via its own input interface. For example, the terminal apparatus 31 is accepts various operation inputs from the physician and transmits the various operation inputs to the biological information processing apparatus 500. the terminal apparatus 31 is implemented as, for example, a PC, a tablet PC, PDA, a mobile phone (smartphone, and the like).

The biological information processing apparatus 500 is an apparatus operated by an administrator of the diagnostic support system 1 and executes various processes to assist a physician in diagnosing the subject. Specifically, the biological information processing apparatus 500 presents information indicating abnormality in the correlation relationship between vital sign data of the subject based on a plurality of vital sign data acquired by the vital sign data acquisition apparatus 21.

The biological information processing apparatus 500 includes, as illustrated in FIG. 1, a communication interface 51, an input interface 52, a display 53, a memory 54, and processing circuitry 55. For example, the biological information processing apparatus 500 is implemented as a computer apparatus such as a PC, a workstation, or a server.

The communication interface 51 controls transmission and communication of various data transmitted and received between the biological information processing apparatus 500 and each apparatus connected via the network 400.

Specifically, the communication interface 51 is connected to the processing circuitry 55 and transmits data received from each apparatus on the network 400 to the processing circuitry 55 or data received from the processing circuitry 55 to each apparatus on the network 400. For example, the communication interface 51 is implemented as a network card, network adapter, NIC, or the like.

The input interface 52 accepts input operations of various instructions and various pieces of information from an operator. Specifically, the input interface 52 is connected to the processing circuitry 55, converts the input operations received from the operator into electrical signals and transmits the signals to the processing circuitry 55.

For example, the input interface 52 can be implemented as a trackball, a switch button, a mouse, a keyboard, a touchpad for input operation by touching an operation surface, a touchscreen in which a display screen and a touchpad are integrated, a non-contact input interface using optical sensors, and a voice input interface, or the like.

In the present specification, the input interface 52 is not limited only to those with physical operating components such as a mouse and a keyboard. For example, processing circuitry for electrical signals that receives electrical signals corresponding to input operations from an external input apparatus installed separately from the apparatus and transmits these electrical signals to a control circuit is also included in the example of the input interface 52.

The display 53 displays various pieces of information and data. Specifically, the display 53 is connected to the processing circuitry 55 and displays various pieces of information and data received from the processing circuitry 55. For example, the display 53 can be implemented as an LCD display, a cathode ray tube (CRT) display, a touch panel, or the like.

The memory 54 stores various data and computer programs. For example, the memory 54 stores a generative model 541 and a medical guideline 542. The configurations of the generative model 541 and the medical guideline 542 are similar to those of the generative model 141 and the medical guideline 142, and the descriptions thereof will not be repeated.

Specifically, the memory 54 is connected to the processing circuitry 55 and stores data received from the processing circuitry 55, or reads and transmits the stored data to the processing circuitry 55.

For example, the memory 54 is implemented as a semiconductor memory element such as a ROM, a RAM, a flash memory, or the like, an HDD, an SSD, an optical disk, or the like. The memory 54 may be implemented as a cloud computer connected to the biological information processing apparatus 500 via the network 400.

The processing circuitry 55 provides overall control of the biological information processing apparatus 500. Specifically, the processing circuitry 55 performs various processes to assist the physician in diagnosing the subject. For example, the processing circuitry 55 controls data exchange with apparatuses on the network 400, data storage in the memory 54, and various processes using the data.

For example, as illustrated in FIG. 12, in the present embodiment, the processing circuitry 55 performs an acquisition function 551, a calculation function 552, an analysis function 553, and a presentation function 554.

The acquisition function 551 acquires a plurality of vital sign data with different types about the subject. For example, the acquisition function 551 acquires vital sign data that is periodically acquired by the vital sign data acquisition apparatus 21 from the terminal apparatus 22 of the subject-side apparatus 200.

The configurations of the calculation function 552, the analysis function 553, and the presentation function 554 is similar to those of the calculation function 163, the analysis function 164, and the presentation function 165, and the descriptions thereof will not be repeated.

The processing circuitry 55 described above is implemented as, for example, a processor. In that case, each processing function described above is stored in the memory 54 in the form of a computer program executable by a computer. Then, the processing circuitry 55 reads and executes each computer program stored in the memory 54 to implement the function corresponding to each computer program. In other words, the processing circuitry 55 has each processing function illustrated in FIG. 1 with each computer program read out.

The processing circuitry 55 may include a plurality of independent processors, each of which executes a computer program to implement each processing function. The processing functions that the processing circuitry 55 has may be distributed or integrated into a single or a plurality of processing circuitries as appropriate. Each processing function that the processing circuitry 55 has may be implemented in combination of hardware such as circuits and software.

Although the example in which the computer program corresponding to each processing function is stored in a single memory 54 is described herein, the embodiment is not limited thereto. For example, computer programs corresponding to processing functions may be distributed and stored in a plurality of memories, and the processing circuitry 55 may read and execute each program from each memory.

Some of the processing functions that the processing circuitry 55 has may also be implemented by a cloud computer connected to the biological information processing apparatus 500 via the network 400.

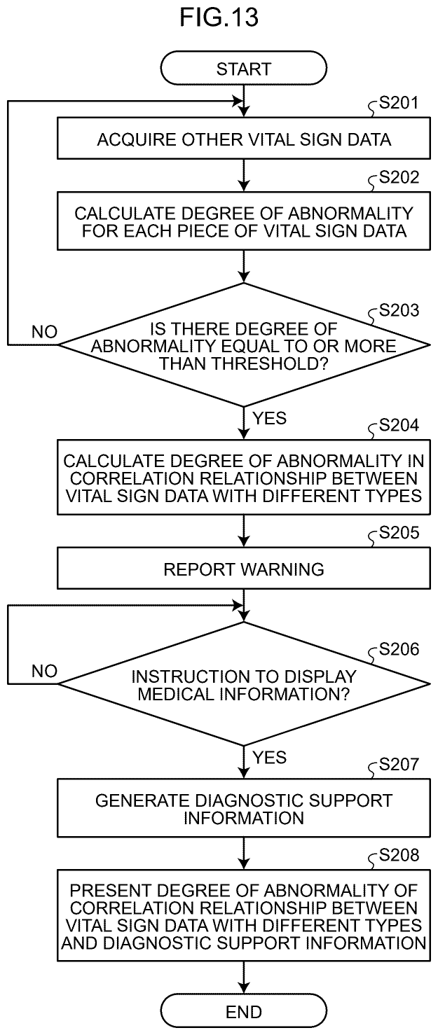

Next, a process executed by the biological information processing apparatus 500 will be described. FIG. 13 is a flowchart illustrating an example of processing performed by the biological information processing apparatus 500 according to the second embodiment.

First, the acquisition function 551 acquires vital sign data of the target subject (step S201). For example, the acquisition function 551 acquires a plurality of vital sign data with different types, which are periodically transmitted from the terminal apparatus 22 of the subject-side apparatus 200a of the target subject.

The processes at steps S202 to S208 are similar to steps S104 to S110 in FIG. 11, and thus the descriptions will not be repeated.

As described above, the biological information processing apparatus 500 of the second embodiment, similar to the weight measurement apparatus 100 of the first embodiment, can assist the user to easily understand the state of the target subject.

The embodiment described above can also be implemented with changes of a part of the configuration or function of each apparatus in the diagnostic support system 1 (1a), as appropriate. Therefore, hereinbelow, modifications according to the above-described embodiments will be described as other embodiments. The following mainly describes the differences from the above-described embodiments, and the detailed descriptions of the points in common with those already described will not be repeated. The modifications described below may be implemented individually or in combination as appropriate.

### First Modification

In the first and second embodiments described above, it has been described for the presentation function 165 (554) to have the form in which the degree of abnormality in the correlation relationship between the vital sign data with different types is displayed as a vertical bar graph. The present modification describes a form of displaying the degree of abnormality of the correlation relationship between the vital sign data with different types in other formation than the vertical bar graph.

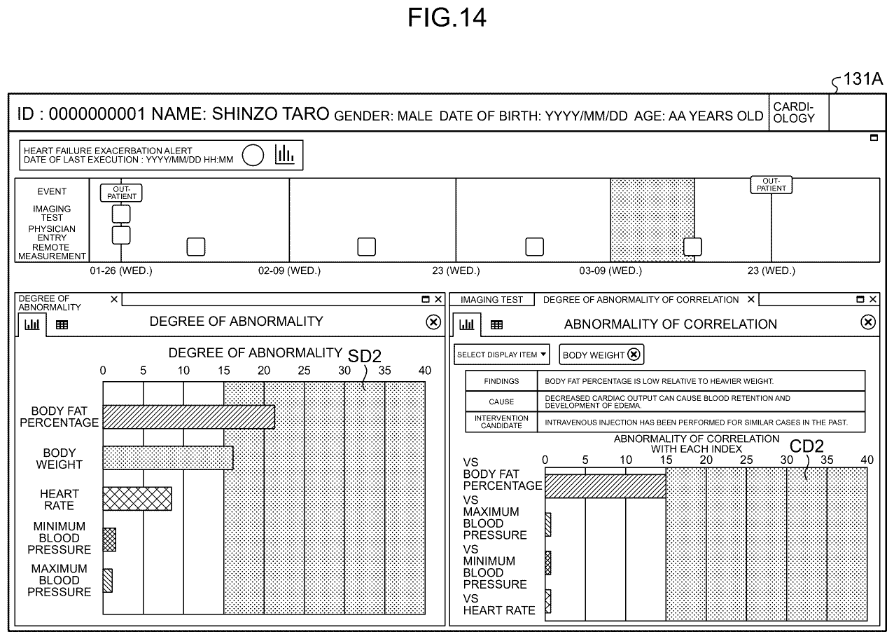

For example, the presentation function 165 (554) may display the degree of abnormality of the correlation relationship between the vital sign data with different types in a horizontal bar graph. FIG. 14 is a diagram for describing an example of a process of presenting the degree of abnormality in the correlation relationship between vital sign data with different types according to the first modification. In FIG. 14, the numerical references of the same parts as in FIG. 10 are not described.

In the example of FIG. 14, a presentation screen 131A has an abnormality degree graph SD2 and a correlation abnormality degree graph CD2 as a screen configuration different from FIG. 10.

The abnormality degree graph SD2 is a horizontal bar graph representing the degree of abnormality for each piece of vital sign data. In the example in FIG. 14, the horizontal axis represents the degree of abnormality for each piece of vital sign data, and the vertical axis represents the types of vital sign data.

In addition, the correlation abnormality degree graph CD2 is a horizontal bar graph representing the degree of abnormality in the correlation relationship between the vital sign data with different types. In the example in FIG. 14, the horizontal axis represents the degree of abnormality in the correlation relationship between the type of vital sign data corresponding to the vital sign tab VT (see FIG. 10) and other types of vital sign data, and the vertical axis represents other types of vital sign data.

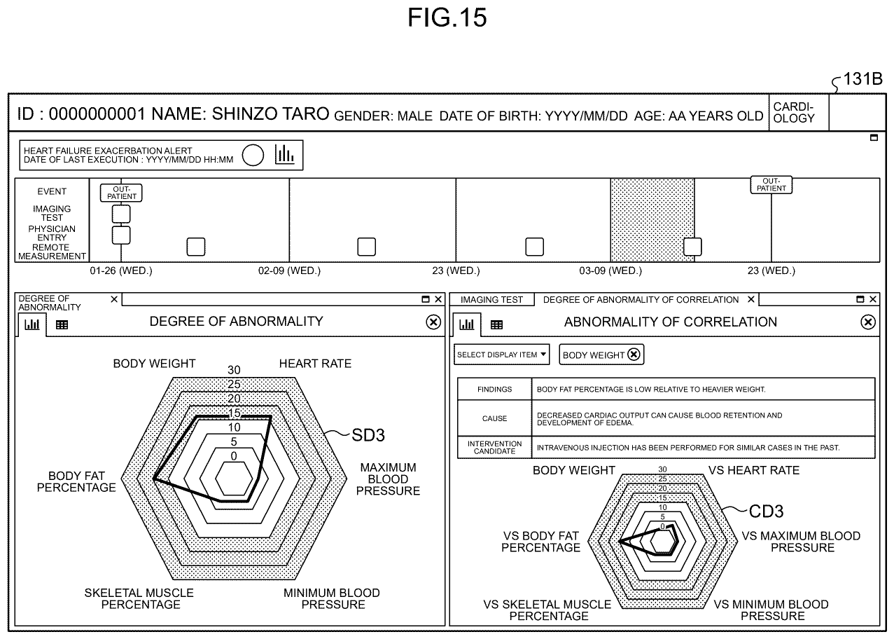

In addition, for example, the presentation function 165 (554) may display the degree of abnormality of the correlation relationship between the vital sign data with different types in a radar chart (chart graph). FIG. 15 is a diagram for describing an example of a process of presenting the degree of abnormality in the correlation relationship between vital sign data with different types according to the first modification. In FIG. 15, the numerical references of the same parts as in FIG. 10 are not described.

In the example of FIG. 15, a presentation screen 131B has an abnormality degree graph SD3 and a correlation abnormality degree graph CD3 as a screen configuration different from FIG. 10.

The abnormality degree graph SD3 is a chart graph representing the degree of abnormality for each piece of vital sign data. In the example in FIG. 15, the types of vital sign data are displayed outside of the abnormality degree graph SD3. In addition, the abnormality degree graph SD3 represents that the outward direction indicates a higher abnormality for each piece of vital sign data.

In addition, the correlation abnormality degree graph CD3 is a chart graph representing the degree of abnormality in the correlation relationship between the vital sign data with different types. In the example in FIG. 15, other types of vital sign data are displayed outside of the correlation abnormality degree graph CD3. The correlation abnormality degree graph CD3 represents that the outward direction indicates a higher degree of abnormality in the correlation relationship between the type of vital sign data corresponding to the vital sign tab VT (see FIG. 10) and other types of vital sign data.

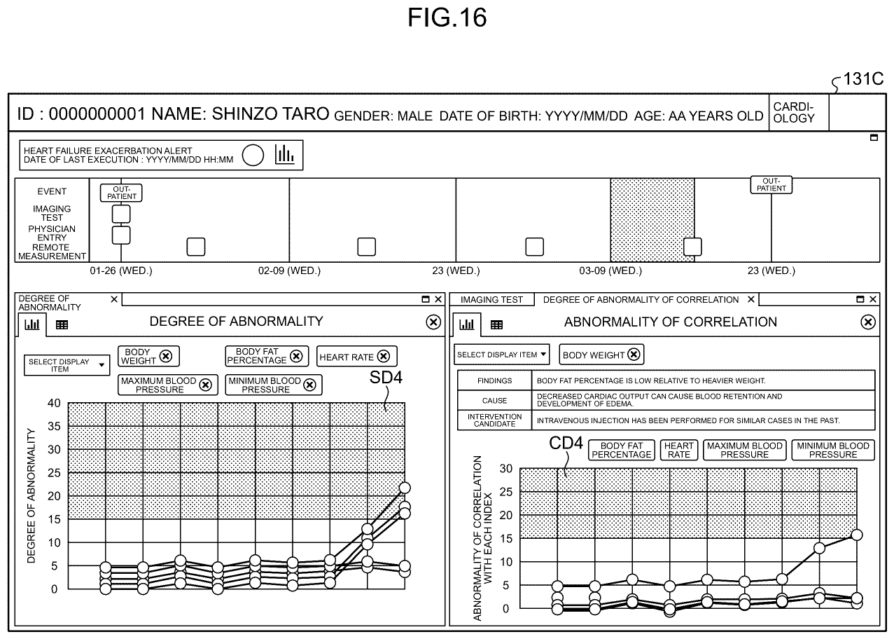

For example, the presentation function 165 (554) may display the degree of abnormality of the correlation relationship between the vital sign data with different types in a line graph. FIG. 16 is a diagram for describing an example of a process of presenting the degree of abnormality in the correlation relationship between vital sign data with different types according to the first modification. In FIG. 16, the numerical references of the same parts as in FIG. 10 are not described.

In the example of FIG. 16, a presentation screen 131C has an abnormality degree graph SD4 and a correlation abnormality degree graph CD4 as a screen configuration different from FIG. 10.

The abnormality degree graph SD4 is a line graph representing the degree of abnormality for each piece of vital sign data. In the example in FIG. 16, the vertical axis represents the degree of abnormality for each piece of vital sign data. The horizontal axis represents time (date).

In addition, in the abnormality degree graph SD4, a plurality of line graphs are displayed. The line graphs each represent a type of vital sign data. In the example in FIG. 16, the user can see the degree of abnormality for each piece of vital sign data in a time series.

The correlation abnormality degree graph CD4 is a line graph representing the degree of abnormality in the correlation relationship between the vital sign data with different types. In the example in FIG. 16, the vertical axis represents the degree of abnormality in the correlation relationship between the type of vital sign data corresponding to the vital sign tab VT (see FIG. 10) and other types of vital sign data. The horizontal axis represents time (date).

In addition, in the correlation abnormality degree graph CD4, a plurality of line graphs are displayed. The line graphs each represent other type of vital sign data. In the example in FIG. 16, the user can see the degree of abnormality in the correlation relationship between the vital sign data with different types in a time series.

The presentation function 165 (554) may display any of the presentation screens 131A to 131C illustrated in FIGS. 14 to 16 instead of the presentation screen 131 in FIG. 10, or may display any of the presentation screens 131, and 131A to 131C according to user instructions. The presentation function 165 (554) may also present the degree of abnormality for each piece of vital sign data and the degree of abnormality in the correlation relationship between the vital sign data with different types in an aspect other than the presentation screens 131, and 131A to 131C described above.

According to the present modification, the degree of abnormality for each piece of vital sign data of the target subject and the degree of abnormality in the correlation relationship between the vital sign data with different types of the target subject can be presented in the aspect that is easy for the user to understand.

### Second Modification

In the first and second embodiments described above, the form in which the biological information is vital sign data was described. The present modification describes a form in which biological information includes information other than vital sign data.

As an example, the biological information may include a measurement value of blood glucose measured by a simple blood glucose meter. In this case, the correlation relationship between different types of biological information may be the correlation relationship between the measurement value of blood glucose and each type of vital sign data.

As another example, the biological information may include measurement values of various subject tests (blood test, urine test, and the like) of the target subject. In this case, the correlation relationship between different types of biological information may be the correlation relationship between the measurement values of various subject tests and each type of vital sign data, or the correlation relationship between the measurement values of different types of subject tests.

According to the present modification, the user can easily identify the abnormality in correlation relationship between different types of biological information, even for biological information other than vital sign data.

In the embodiments described above, the example of the case in which each processing function in the present specification is implemented by single processing circuitry is described, but the embodiments are not limited thereto. For example, the embodiments may implement each processing function in the present specification using only hardware or software, or using combination of hardware and software.

The term “processor” used in the description of the above-described embodiments refers to, for example, a central processing unit (CPU), a graphics processing unit (GPU), or circuitry such as an application specific integrated circuit (ASIC), and a programmable logic device (for example, simple programmable logic device (SPLD), complex programmable logic device (CPLD), and field programmable gate array (FPGA)).

Here, instead of storing a computer program in the memory, the computer program can be configured to be incorporated directly into the circuit of the processor. In this case, the processor reads and executes the computer program embedded in the circuit to implement the function. Each processor in the present embodiment is not limited to the case where each processor is configured as a single circuit, but may also be configured as a single processor by combining a plurality of independent circuits to implement its functions.

Here, a supporting computer program to be executed by the processor is provided pre-embedded in a read only memory (ROM), a memory, or the like. This supporting computer program may be provided in a format that can be installed on these apparatuses or as a file in an executable format and recorded on a compact disk-(CD)-ROM, flexible disk (FD), compact disk recordable (CD-R), digital versatile disk (DVD), or other computer-readable non-transitory storage medium.

This supporting computer program may also be provided or distributed by being stored on a computer connected to a network such as the Internet and downloaded over the network. For example, this supporting computer program includes modules containing each of the processing functions described above. As for the actual hardware, the CPU reads and executes the medical image processing program from the storage medium such as ROM, and each module is loaded onto the main memory and generated on the main memory.

In the embodiments and modifications described above, each component of each apparatus illustrated in the figures is a functional concept, and is not necessary to be physically configured as illustrated in the figures. In other words, the specific form of dispersion or integration of each apparatus is not limited to that illustrated in the figure, but can be configured by functionally or physically dispersing or integrating all or part thereof in any units, depending on various loads and usage conditions.

Furthermore, the whole or part of each processing function performed by each apparatus can be implemented by CPU and a computer program that is analyzed and executed by the CPU, or as hardware using wired logic.

In the above-described embodiments and modifications, all or some of the processes described as being performed automatically may instead be performed manually, and otherwise, all or some of the processes described as being performed manually may instead be performed automatically using known methods. Other information including processing procedures, control procedures, specific names, and various data and parameters illustrated in the above documents and drawings may be changed as desired, unless otherwise specified.

According to at least one of the embodiments described above, it is possible to assist the non-physician healthcare professionals to easily understand the state of the subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A weight measurement apparatus comprising processing circuitry configured to:
acquire a body weight of a subject and biological information other than the body weight;
calculate a degree of abnormality of the body weight based on a predetermined threshold;
calculate a degree of abnormality of a correlation based on a correlation relationship between the body weight and the biological information; and
output information indicating the degree of abnormality in the body weight and information indicating a degree of deviation between the correlation relationship used in the calculating of the degree of abnormality in the correlation and a reference correlation relationship.

2. A biological information processing apparatus comprising processing circuitry configured to:
acquire a plurality of pieces of biological information with different types about a subject;
calculate a degree of abnormality of first biological information included in the plurality of pieces of biological information based on a correlation relationship with a second biological information included in the plurality of pieces of biological information other than the first biological information; and
output, for each piece of the second biological information, information indicating a degree of deviation between the correlation relationship used in the calculating of the degree of abnormality and a reference correlation relationship.

3. The biological information processing apparatus according to claim 2, wherein
the processing circuitry configured to:
generate a finding for the degree of abnormality; and
cause a display apparatus to display the finding together with the information indicating the degree of deviation between the correlation relationship in which the degree of abnormality of the first biological information is used to calculate the degree of abnormality for each piece of the second biological information, and the reference correlation relationship.

4. The biological information processing apparatus according to claim 3, wherein
the processing circuitry configured to generate the finding by:
deriving, based on a calculation result of the degree of abnormality of the first biological information and the correlation relationship used in the calculating of the degree of abnormality of the first biological information, a state of the first biological information for each type of the first biological information and a state of the second biological information for each type of the second biological information; and
populating a formulated phrase configured to be fillable and to express a correlation relationship between the first biological information and the second biological information with each type of the first biological information and the state of the first biological information, and each type of the second biological information and the state of the second biological information.

5. The biological information processing apparatus according to claim 3, wherein
the processing circuitry is configured to:
generate, based on a calculation result of the degree of abnormality of the first biological information and the finding, information related to a cause leading the first biological information to indicate the degree of abnormality of the first biological information; and
display, on the display apparatus, the information related to the cause together with the information indicating a degree of deviation between the correlation relationship in which the degree of abnormality of the first biological information is used to calculate the degree of abnormality for each type of the second biological information and the reference correlation relationship.

6. The biological information processing apparatus according to claim 5, wherein
when an instruction sentence is input, the processing circuitry is configured to
input the instruction sentence generated based on the finding to a large language model (LLM) functionally configured to generate and output a response sentence regarding the information related to the cause in accordance with a meaning of the input instruction sentence, and
display, on the display apparatus, the response sentence output from the LLM as the information related to the cause.

7. The biological information processing apparatus according to claim 6, wherein
the processing circuitry is configured to generate the instruction sentence including words included in the finding and included in a medical guideline representing a guideline compiling information on a rationale and a procedure for medical treatment, such as disease prevention, diagnosis, treatment, and prognosis prediction.

8. The biological information processing apparatus according to claim 3, wherein
the processing circuitry is configured to
generate a candidate for intervention to reduce the degree of abnormality based on a calculation result of the degree of abnormality and the finding, and
display the candidate for intervention on the display apparatus.

9. The biological information processing apparatus according to claim 8, wherein
when an instruction sentence is input, the processing circuitry is configured to
input the instruction sentence generated based on the finding to LLM functionally configured to generate and output a response sentence regarding the candidate for intervention in accordance with a meaning of the input instruction sentence, and
display, on the display apparatus, the response sentence output from the LLM as the candidate for intervention.

10. The biological information processing apparatus according to claim 9, wherein
the processing circuitry is configured to generate the instruction sentence including words included in the finding and included in a medical guideline representing a guideline compiling information on a rationale and a procedure for medical treatment, such as disease prevention, diagnosis, treatment, and prognosis prediction.

11. The biological information processing apparatus according to claim 3, wherein
the processing circuitry is configured to represent and display the information indicating the degree of deviation between the correlation relationship used in the calculating of the degree of abnormality and the reference correlation relationship using at least one of a vertical bar graph, a horizontal bar graph, a radar chart, or a line graph in a time series, on the display apparatus.

12. The biological information processing apparatus according to claim 2, wherein
the processing circuitry is configured to
receive an input for selecting a type of the biological information serving as the first biological information among the types of the biological information,
define the selected type of the biological information as the first biological information, and
output the information indicating the degree of abnormality of the first biological information, and the information indicating the degree of deviation between the correlation relationship used in the calculating of the degree of abnormality for each piece of the second biological information and the reference correlation relationship.

13. A biological information processing method comprising:
acquiring a plurality of pieces of biological information with different types about a subject;
calculating a degree of abnormality of first biological information included in the plurality of pieces of biological information based on a correlation relationship with a second biological information included in the plurality of pieces of biological information other than the first biological information; and
outputting, for each piece of the second biological information, information indicating a degree of deviation between the correlation relationship used in the calculating of the degree of abnormality and a reference correlation relationship.
